(19) United States
(12) Patent Application Publication
(10) Pub. No.: US 2011/0230473 A1
(43) Pub. Date: Sep. 22, 2011

(54) Methods and Compositions for Treating Status Epilepticus and Seizures Causing Status Epilepticus

(76) Inventors: Richard K. Gordon, Potomac, MD (US); Madhusoodana P. Nambiar, Mt. Airy, MD (US); James C. Demar, Rockville, MD (US); Ruthie H. Ratcliffe, Glen Burnie, MD (US); Bhupendra P. Doctor, Potomac, MD (US); Roberta R. Owens, Silver Spring, MD (US)

(21) Appl. No.: 13/122,901

(22) PCT Filed: Oct. 9, 2009
(86) PCT No.: PCT/US2009/060091

§ 371 Date: May 17, 2011

Related U.S. Application Data
(60) Provisional application No. 61/104,388, filed on Oct. 10, 2008.
(60) Provisional application No. 61/104,311, filed on Oct. 10, 2008.

(51) Int. Cl. A61K 31/5513; A61K 31/439; A61K 31/44; A61P 25/08

(52) U.S. Cl. 514/221; 514/295; 514/357

(57) ABSTRACT

Disclosed herein are methods, kits and compositions for treating, preventing, inhibiting, or reducing a seizure, status epilepticus, neuropathogenesis or a neuropathology caused by overstimulation of the NMDA receptor pathway and/or exposure to an OP compound.

### CROSS-REFERENCE TO RELATED APPLICATIONS

This application claims the benefit of U.S. APPLICATION Ser. No. 61/104,388, filed 10 Oct. 2008, and U.S. Application Ser. No. 61/104,311, filed 10 Oct. 2008, both of which are herein incorporated by reference in their entirety.

### ACKNOWLEDGEMENT OF GOVERNMENT SUPPORT

This invention was made by employees of the United States Army Medical Research and Materiel Command. The Government has rights in this invention.

### BACKGROUND OF THE INVENTION

1. Field of the Invention

The present invention generally relates methods and compositions for treating seizures, seizures which cause status epilepticus, status epilepticus, and neuropathogenesis caused by cholinesterase inhibitors. The present invention also generally relates methods and compositions for treating seizures, seizures which cause status epilepticus, status epilepticus, and neuropathogenesis caused by overstimulation of the NMDA receptor pathway.

2. Description of the Related Art

Organophosphate (OP) compounds inhibit the catalytic sites of cholinesterases (ChE), such as acetylcholinesterase (AChE) and butyrylcholinesterase (BChE). Inhibition of AChE leads to a build up of acetylcholine (ACh) in the central nervous system (CNS) and peripheral nervous system (PNS) that disrupts cholinergic neurotransmission. Exposure to OP compounds can induce seizures. If the seizures persist, neuropathogenesis (which leads to neuropathology, e.g. neuronal damage) and status epilepticus (SE) may result. SE is characterized by prolonged epileptic seizures that can produce long-term CNS damage and behavioral alterations in survivors and can cause death if untreated. It should be noted that not all OP compounds result in seizures or SE. In addition, some OP compounds may result in SE at one amount, but not another. See e.g. Crawford et al. (2004) (published online at handle.dtic.mil/100.2/ADA449679).

The mechanisms of OP induced seizures are generally divided into three phases. See McDonough & Shih (1997) Neurosci Biobehav Rev 21:559-579; and Carpentier (2008) J Med CBR Def 6 (published online). The first phase involves cholinergic based mechanisms, i.e. changes in brain AChE and accumulation of ACh, which begins from the time of exposure to about 5 min after seizure onset. The second phase is a transitional phase which is a combination of cholinergic and non-cholinergic based mechanisms, wherein excitatory amino acids (EAA) and glutamate are released, which over-stimulate N-methyl-D-aspartate (NMDA) receptors. The third phase comprises predominantly non-cholingeric based mechanisms. If seizure activity is not stopped before increased concentrations of glutamate result in glutamate neurotoxicity progression to SE often occurs.

Currently, seizures are treated with benzodiazepines, phenyloin, fosphenyloin, barbituates, and/or anesthetics. However, many of these treatments are ineffective against seizures induced by OP compounds and nerve agents.

### SUMMARY OF THE INVENTION

The present invention provides a method of treating, preventing, inhibiting, or reducing a seizure, such as a SE causing seizure, status epilepticus, neuropathogenesis, or a neuropathology caused by exposure to an organophosphate compound in a subject in need thereof which comprises administering to the subject Pro-2-PAM, a huperzine compound, or both. In some embodiments, the present invention is directed to a method of increasing the survivability of a subject exposed, such as by cutaneous exposure, to an organophosphate compound which comprises administering to the subject Pro-2-PAM, a huperzine compound, or both. In some embodiments, Pro-2-PAM and/or the huperzine compound is administered before, during or after exposure to the organophosphate compound. In some embodiments, Pro-2-PAM and the huperzine compound are administered at the same time, different times, or both. In some embodiments, the huperzine compound is administered as an enantiopure composition or as a mixture.

In some embodiments, administration of Pro-2-PAM and/or the huperzine compound suppresses, eliminates, or protects the subject against seizure activity, seizures, such as an SE causing seizure, status epilepticus, neuropathogenesis, or a neuropathology caused by exposure to an organophosphate compound. In some embodiments, administration of Pro-2-PAM and/or the huperzine compound restores brain AChE activity.

In some embodiments, the present invention is directed to treating, preventing, inhibiting, or reducing a seizure, such as a SE causing seizure, status epilepticus, neuropathogenesis, or a neuropathology caused by exposure to an organophosphate compound in a subject in need thereof which comprises reactivating the extracellular AChE in the brain of the subject by administering Pro-2-PAM to the subject.

In some embodiments, the present invention provides a kit which comprises Pro-2-PAM and the huperzine compound packaged together. In some embodiments, the kit further comprises at least one device, such as an autoinjector, for delivering Pro-2-PAM, the huperzine compound, or both to a subject. In some embodiments, the autoinjector comprises a first compartment containing Pro-2-PAM and a second compartment containing the huperzine compound.

In some embodiments, the present invention provides a composition comprising Pro-2-PAM and the huperzine compound.

In some embodiments, the present invention provides a method of treating, preventing, inhibiting, or reducing a seizure, such as a SE causing seizure, status epilepticus, neuropathogenesis, or a neuropathology caused by overstimulation of the NMDA receptor pathway in a subject in need thereof which comprises administering to the subject a huperzine compound. In some embodiments, the huperzine compound is administered before, during or after the NMDA receptor pathway is overstimulated. In some embodiments, the huperzine compound is administered as an enantiopure composition or as a mixture. In some embodiments, the overstimulation of the NMDA receptor pathway is caused by a brain injury such as a penetrating traumatic brain injury (e.g. those caused by bullets, shrapnel, etc.) or a blast induced traumatic brain injury (i.e. closed head injury, e.g. those caused by bombs).

In some embodiments, the present invention is directed to a medicament for treating, preventing, inhibiting, or reducing a seizure, such as a SE causing seizure, status epilepticus, neuropathogenesis, or a neuropathology which comprises Pro-2-PAM and/or a huperzine compound. In some embodiments, the seizure, the SE causing seizure, SE, neuropathogenesis, or the neuropathology is caused by overstimulation of the NMDA receptor pathway or exposure to an OP compound. In some embodiments the overstimulation of the NMDA receptor pathway is caused by a brain injury.

In the above embodiments and other embodiments as disclosed herein, the huperzine compound may be a huperzine A compound, preferably +HupA. In the above embodiments and other embodiments as disclosed herein, Pro-2-PAM, the huperzine compound, or both may be provided as a single dose or multiple doses. In the above embodiments and other embodiments as disclosed herein, Pro-2-PAM and the huperzine compound are provided in therapeutically effective amounts. In some embodiments, the therapeutically effective amounts are amounts which treat, prevent, inhibit, or reduce a seizure, an SE causing seizure, status epilepticus, neuropathogenesis, or a neuropathology caused by exposure to an organophosphate compound as compared to a control. In some embodiments where the seizure, the SE causing seizure, status epilepticus, neuropathogenesis, or the neuropathology is caused by overstimulation of the NMDA receptor pathway not involving exposure to an OP compound, such as a brain injury, therapeutically effective amounts of the huperzine compound are ones which treat, prevent, inhibit, or reduce a seizure, an SE causing seizure, status epilepticus, neuropathogenesis, or a neuropathology as compared to a control. In the above embodiments and other embodiments as disclosed herein, 2-PAM, a second huperzine compound, a supplementary active compound, or a combination thereof may be administered.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIG. 1A, Panel A shows EEG data that is representative of a saline control rat administered only PB and saline. Panel B shows EEG data for a DFP control animal. Each line represents one hour of EEG recording and 12 hr of recordings are shown. The scale on the Y-axis is −1 mV to +1 mV. Time and dose of drugs administered are indicated with arrows.

FIG. 1B shows a 10 sec segment of EEG data from one saline control animal. It is taken from 3 hr after saline administration. Y-axis scale is −1 mV to +1 mV.

FIG. 1C shows a 10 sec segment of EEG data from one DFP exposed control animal. It is taken from 3 hr after DFP administration. Y-axis scale is −1 mV to +1 mV.

FIG. 2A shows EEG data that is representative of a rat pre-treated with +HupA and then exposed to DFP. Each line represents 1 hr of EEG recording and 12 hr of recordings are shown. The scale on the Y-axis is −1 mV to +1 mV. Time and dose of drugs administered are indicated with arrows.

FIG. 2B is a 10 sec segment of EEG data from one animal pre-treated with +HupA is shown here. It is taken from 3 hr after DFP administration. Y-axis scale is −1 mV to +1 mV.

FIG. 3A shows EEG data for a rat treated with +HupA 1 min after DFP exposure. Each line represents 1 hr of EEG recording and 12 hr of recordings are shown. The scale on the Y-axis is −1 mV to +1 mV. Time and dose of drugs administered are indicated with arrows.

FIG. 3B is a 10 sec segment of EEG data from one rat treated with +HupA 1 min after DFP exposure. It is taken from 3 hr after DFP administration. Y-axis scale is −1 mV to +1 mV.

FIG. 4A shows EEG data for a rat treated with +HupA 5 min after DFP exposure. Each line represents 1 hr of EEG recording and 12 hr of recordings are shown. The scale on the Y-axis is −1 mV to +1 mV. Time and dose of drugs administered are indicated with arrows.

FIG. 4B is a 10 sec segment of EEG data from one rat treated with +HupA 5 min after DFP exposure. It is taken from 3 hr after DFP administration. Y-axis scale is −1 mV to +1 mV.

FIG. 5A shows EEG data for one rat treated with +HupA 10 min after DFP exposure. Each line represents 1 hr of EEG recording and 12 hr of recordings are shown. The scale on the Y-axis is −1 mV to +1 mV. Time and dose of drugs administered are indicated with arrows.

FIG. 5B is a 10 sec segment of EEG data from one rat treated with +HupA 10 min after DFP exposure. It is taken from 3 hr after DFP administration. Y-axis scale is −1 mV to +1 mV.

FIG. 6 shows representative EEG traces for Control, DFP, DFP then 2-PAM, and DFP then Pro-2-PAM treated guinea pigs. Animals received the standard military paradigm, e.g. 2-PAM 1 min post-exposure except for Pro-2-PAM treatment which was delayed by 15 min. Each line of EEG data represents 2 hr of recording. Thus, a full 24 hr are shown (12 lines) for each animal treatment. Solid circle indicates time of DFP exposure.

FIG. 7 shows H&E stains of guinea pig brain samples at 40× magnification of the piriform cortical neuron layer. Panel (a) is Control brain; Panel (b) is brain from animal receiving DFP only; Panel (c) is brain from animal receiving DFP followed by Pro-2-PAM; and Panel (d) is brain from animal receiving DFP followed by 2-PAM. Arrows point to piriform neurons.

FIG. 8 shows fluoro-jade stains of guinea pig brain samples at 40× magnification, of the hippocampus pyramidal neuron layer. Panel (a) is Control brain; Panel (b) is brain from animal receiving DFP followed by 2-PAM; and Panel (c) is brain from animal receiving DFP then Pro-2-PAM. Arrows point to hippocampus pyramidal neurons.

FIG. 9 shows blood AChE activity (U/ml) at 1.5 hr post treatment. These data show 2-PAM and pro-2-PAM equivalence for peripheral reactivation of DFP-inhibited AChE. Numbers in brackets are animals tested.

FIG. 10 shows AChE activity (mU/mg) in eight specific brain regions from guinea pigs 1.5 hr after treatment with saline (Control, ), PB (▾), DFP only (□), or animals treated with DFP followed by 2-PAM (▪) or pro-2-PAM (▴). The number of animals is in brackets. * indicates significant difference between Pro-2-PAM and 2-PAM treatments; p≦0.05.

FIG. 11 shows brain (frontal cortex) AChE activity (mU/mg) at 24 hr after treatment with Pro-2-PAM at indicated times after DFP exposure. These data show that Pro-2-PAM reactivated DFP-inhibited brain AChE when given up to 40 min post-OP exposure. Numbers above points are number of animals tested. Dashed lines are average AChE activity for Pro-2-PAM treated animals (black dashed) compared to DFP only animals (gray dashed). * indicates significant difference between Pro-2-PAM treatment and DFP without oxime treated animals; p≦0.05.

FIG. 12 provides the structural formulas of examples of huperzine A compounds which may be administered in place of or in conjunction with +HupA. Huperzine A compounds TSK-V-3 [−]-19, TSK-V-4 [+]-19, TSK-IV-90B [−]-18, TSK-IV-78B and [+]dimethylhuperzine showed protective efficacy against NMDA and DFP toxicity in cell culture. Huperzine A compounds TSK-V-3 [−]-19, TSK-V-4 [+]-19, and TSK-IV-90B [−]-18, were found to be protective against neuropathology resulting from exposure to DFP and soman.

### DETAILED DESCRIPTION OF THE INVENTION

The present invention is directed to methods and compositions for treating, preventing, inhibiting, or reducing seizures, status epilepticus causing seizures, status epilepticus (SE), neuropathogenesis, and neuropathologies caused by exposure to a cholinesterase inhibitor which comprise administering to a subject in need thereof a therapeutically effective amount of Pro-2-PAM, a Huperzine compound, or both. The present invention is also directed to methods and compositions for treating, preventing, inhibiting, or reducing seizures, SE causing seizures, SE, neuropathogenesis, and neuropathologies caused by overstimulation of the NMDA receptor pathway, which overstimulation may be caused by a brain injury, which comprise administering to a subject in need thereof a therapeutically effective amount of a Huperzine compound.

As used herein, a “cholinesterase inhibitor” refers to a compound which inhibits a cholinesterase (ChE), e.g. acetylcholinesterase (AChE), from breaking down its substrate, e.g. acetylcholine (ACh). Cholinesterase inhibitors include organophosphate (OP) compounds, diisopropyl-n-fluorophosphate, OP insecticides, such as azinphos-methyl (Gusathion, Guthion), bornyl (Swat), dimefos (Hanane, Pestox XIV), methamidophos (Supracide, Ultracide), methyl parathion (E 601, Penncap-M), chlorpyrifos, Dichloroves, paraoxon, and Demeton S, and OP nerve agents, such as cyclosarin, sarin, soman, tabun, VR, VX, Novichok-5 and Novichok-7, and the like.

As used herein, “status epilepticus” is defined as one continuous unremitting seizure lasting longer than 30 min, or recurrent seizures without regaining consciousness between seizures for greater than 30 min.

As used herein, an “SE causing seizure” are those which lead to SE if not treated, prevented, inhibited or reduced and is typically one that last for more than about 5 min to about 30 min.

As set forth herein, a “huperzine compound” refers to synthetic and natural huperzine compounds known in the art. See e.g. US Pat. Publ. 20080090808. “Huperzine A” (HupA) refers to 9-amino-13-ethylidene-11-methyl-4-azatricyclo[7.3.1.0]trideca-3(8),6,11-trien-5-one. The (−) and (+) enantiomers of HupA are indicated as −HupA and +HupA, respectively. The designation “HupA” refers to −HupA, +HupA, or both. “±HupA” is used to indicate a racemic mixture of +HupA and −HupA. The phrase “huperzine A compound” refers to HupA and analogs, derivatives, salts, hydrates, homologs, positional isomers, and stereoisomers thereof. Examples of huperzine A compounds include those set forth in U.S. Pat. Nos. 4,929,731; 5,106,979; 5,663,344; and 5,869,672; 5,104,880; 5,177,082; 5,929,084; and 5,547,960; dihydro-desmethyl-huperzine; 11-desmethyl-11-chloro-huperzine A, those shown in FIG. 12, and the like. Preferred huperzine compounds do not result in cardiotoxicity.

As used herein, “Pro-2-PAM” refers to N-methyl-1,6-dihydropyridine-2-carbaldoxime and salts and solvates thereof. Pro-2-PAM may be synthesized using methods known in the art. See e.g. Bodor (1976) J. Med. Chem. 19:102-107. Pro-2-PAM can be stored as a readily water soluble powder, similar to the oxime HI-6, and administered using methods and devices known in the art.

As used herein, a “subject” includes animal subjects and human subjects. A subject is considered to be “in need” of the treatments and compositions according to the present invention is considered to be a subject exposed to or at risk of exposure to an amount of a cholinesterase inhibitor which amount is likely to result in SE and/or neuropathogenesis if untreated.

As used herein, “neuropathogenesis” refers to the process of neuronal degeneration, seizure, apoptosis, necrosis, aberrant cell signaling, energy depletion, calcium toxicity, excitatory amino acid toxicity, oxidative stress, and inflammation.

As used herein, a “neuropathology” refers to the result of CNS neuropathogenesis such as neuronal damage, neuronal degeneration, neuronal cell death, swollen brain tissue, abnormal brain structures, pyramidal neuron layer disruption, deformed neuronal nuclei, axonal injury, neurobehavioral deficits, and the like.

The phrase “a therapeutically effective amount” refers to an amount of a given drug or compound, e.g. +HupA or Pro-2-PAM, which when administered to a subject is of sufficient quantity to achieve the intended purpose, such as to prevent, reduce or inhibit SE causing seizures, SE, neuropathogenesis, a neuropathology, or a combination thereof caused by overstimulation of the NMDA receptor pathway or exposure to an OP compound. Of course, the actual amount will depend upon a variety of factors including, inter alia, the timing of the administration, the condition being treated, the presence of other concurrent diseases or disorders, the age, weight, and general health of the subject. Determination of a therapeutically effective amount and timing of administration is well within the capabilities of those skilled in the art, especially in light of the detailed disclosure herein and the dosages described herein are exemplary dosages which can be used as a guideline.

### Huperzine A

Huperzine A (HupA) is an alkaloid and a ChE inhibitor which leads to an increase in ACh. −HupA has a much higher affinity for AChE than +HupA. See McKinney et al. (1991) Eur Pharmacol 203:303-305. In particular, +HupA has a 100 to 1000-fold lower AChE binding activity than −HupA. Thus, −HupA is used in the treatment of Alzheimer's Disease.

Pretreatment with +HupA prevents seizures induced by pilocarpine in rat seizure models, but pretreatment with −HupA does not. See Tetz et al. (2006) Toxicol Ind Health 22:255-266. Pilocarpine is a muscarinic alkaloid which is a cholinergic receptor agonist.

−HupA is also an antagonist of NMDA (N-methyl D-aspartate) receptors and therefore protects brain neurons from prolonged excitation from NMDA which can ultimately result in death. See Gordon et al. (2001) J Appl Toxicol 21 (Suppl. 1):S47-51. However, since −HupA and +HupA have different AChE binding activities and the NMDA ion channel has not been modeled and its 3-D structure is unknown, it was unknown and could not be predicted whether +HupA would effectively antagonize NMDA receptors in order to treat, prevent, inhibit or reduce SE causing seizures and SE involving overstimulation of the NMDA receptor pathway (e.g. induced by NMDA, OP compounds, and/or brain injuries). Also, since NMDA antagonists such as phencyclidine are known to produce severe behavioral decrements (Willets J, et al. (1990) Trends Pharmacol Sci 11(10):423-8), it was unknown whether +HupA would present the same side-effects and preclude its use.

### +HupA and SE Causing Seizures and SE Induced by NMDA

The protective efficacy of +HupA for NMDA-induced seizures (i.e. seizures caused by overstimulation of the NMDA receptor pathway) was investigated using a rat model. Generally, rats were implanted with radiotelemetry probes to record electroencephalography (EEG), electrocardiography (ECG), body temperature, and physical activity were administered with various doses of +HupA (intramuscularly, i.m.) and treated with 20 μg/kg NMDA (intracerebroventricularly, i.c.v.).

Male Sprague-Dawley rats (200-250 gm, Rattus norvegicus) were purchased from Charles River Laboratories (Wilmington, Mass.). Rats were housed individually in microisolator cages with a 12 hr light/dark cycle. Food and water were available ad libitum, and a one week stabilization period preceded surgery and experimentation. The radiotelemetry system used included 8 receivers and TL10M3-F50-EET bipotential radiotelemetry probes purchased from Data Sciences International (St. Paul, Minn.). The probes were sterilized using 4% glutaraldehyde and handled as instructed by the manufacturer.

Rats were anesthetized by placing them in a chamber with isoflurane gas (2-5% isoflurane, oxygen 1 L/min flow rate). Anesthetized rats were shaved on the head and back and placed in a stereotax (David Kopf Instruments, Tujunga, Calif.) over a water heating jacket. The mouth and nose of the rat were placed in an adapter connected to a supply of isoflurane gas (2-3% isoflurane, oxygen 1.5 L/min flow rate). The dorsal surfaces of the rat's abdomen and head were cleaned and two small initial incisions were made: one along the midline of the back, 7.5 cm anterior to the tail, and one along the dorsal midline of the head. Two cortical electrodes and a reference electrode of the telemetry probe were tunneled subcutaneously from the posterior (back) incision to the anterior (head) incision. The skull was cleaned with gauze, and any open veins or arteries were closed by surgical cautery. Three 1 mm holes were drilled, and screws were inserted: two screws 3 mm anterior to the lambdoid suture and 3 mm on each side of the sagittal suture, and one screw 3 mm to the right of the sagittal suture and 3 mm anterior to the coronal suture. The reference electrode was attached to the forward-most screw and screwed into place. The positive electrode was placed at the right, posterior screw, and the negative electrode was placed at the left screw. VETBOND (Plastics One, Roanoke, Va.) was used to keep the electrodes in place. The incision was sutured using Ethicon sutures.

Placement of cannula for i.c.v. NMDA administration. The cannula and dummy cannula were obtained from Plastics One (Roanake, Va.). A 1 mm burr hole was made 1 mm posterior and 1.4 mm to the right of the bregma. The cannula was inserted 5 mm below the top of the skull and immobilized using vet bond. A dummy cannula was inserted and screwed in place until NMDA administration. Thus, when NMDA was administered, the animals were gently anesthetized using isoflurane and placed on the stereotaxic equipment. The dummy cannula was unscrewed and a needle connected to a Hamilton microsyringe was inserted. NMDA in a volume of 10 μl was injected, and the dummy cannula was put back into place.

Placement of ECG wires and probe. The positive ECG electrode was subcutaneously tunneled along the left side of the rat's abdomen to the xiphoid process, and the negative ECG wire was subcutaneously tunneled along the right anterior side to the right pectoral muscle. The probe was inserted subcutaneously on the left dorsal pocket of the rat. The incision was sutured, and the rat was injected with bupivacaine to alleviate the discomfort from the surgical procedure.

NMDA SE rat model. Rats were randomly assigned to either an experimental group (n=6) or a control group (n=6). The telemetry probes were activated using an external magnet, and the rat's cage was placed at the center of a telemetry receiver. Radiotelemetry data was monitored continuously beginning 30 min before any treatment. The experimental group was injected with an NMDA dose of 20 μg/kg, i.c.v. which, based on a previous study, induces “popcorn” seizures which cause SE. Rats administered with NMDA showed strong SE causing seizures at about 14-16 min post NMDA treatment which immediately became SE. The survival of animals following NMDA administration was 50% (n=6). EEG recordings showed the seizure voltage increased gradually and reached an average value from +0.7 mV to −0.7 mV within 10 min. The seizure amplitude was even higher (+0.7 mV to −1 mV) at 2.5 hr to 5 hr after NMDA administration. After 5 hr the seizure voltage magnitude started to drop gradually but remained higher than the baseline for the full 24 hr monitoring period. The control group received i.c.v. injections of an equal volume of saline. Behavioral data, such as eating, drinking, mobility, and seizure activity were noted continuously for 4-6 hr after injections. After 24 hr, all surviving rats were euthanized.

Administration of +HupA pre- and post-exposure to NMDA was found to protect animals against SE causing seizures and SE and NMDA-administered animals showed increased survival with +HupA treatment. In particular, (a) 3 mg/kg +HupA i.m. 1 min after 20 μg/kg NMDA i.c.v. reduced SE causing seizures to that substantially similar to saline controls; and (b) 1, 2 and 3 mg/kg +HupA i.m. doses delivered 30 min prior to 20 μg/kg NMDA i.c.v. protected against SE causing seizures and SE induced by NMDA. See Coleman et al. (2008) Chemico-Biological Interactions 175:387-395, and U.S. Application Ser. No. 61/104,388.

AChE Activity. AChE activity can be assayed using methods known in the art. See e.g. Ellman et al. (1961) Biochem Pharmacol 7:88-95; Doctor et al. (1987) Anal Biochem 166:399-403; Bradford (1976) Anal Biochem 72:248-254; and U.S. Pat. No. 6,746,850. AChE activity assays indicated that there was no significant difference between the blood and brain AChE activities of rats pre- and post-exposure treated with +HupA and rats receiving NMDA alone. The lack of blood AChE inhibition indicates that +HupA neuroprotection is mediated by NMDA antagonism and the protective mechanism of +HupA is not due to any effect on AChE.

Thus, these experiments indicate that +HupA may be used to treat, prevent, inhibit or reduce SE causing seizures and SE by blocking NMDA-induced excitotoxicity in vivo. Previous studies suggest that −HupA protects against OP toxicity by the reversible inhibition of AChE. However, this is the first finding that +HupA protects against SE causing seizures and SE sustained by excitatory amino acids (EAAs) which over-stimulate NMDA receptors after inhibition of AChE, i.e. after a cholinesterase inhibitor, such as an OP compound, has inhibited AChE.

Therefore, in some embodiments, the present invention provides methods for treating, preventing, inhibiting, or reducing SE causing seizures and SE caused by overstimulated NMDA receptors in a subject in need thereof which comprises administering to the subject a therapeutically effective amount of a huperzine A compound, such as +HupA. In some embodiments, the huperzine A compound is administered before, during, or after the event, e.g. exposure to a compound which results in increased levels of EAAs, which causes the overstimulation of the NMDA receptors. In some embodiments, the huperzine A compound is administered as an enantiopure composition, e.g. +HupA. In some embodiments, +HupA is administered as a mixture with −HupA in order to additionally provide the protective benefits of the anti-cholinergic activity of −HupA. In some embodiments, the mixture is a racemic mixture. In some embodiments, the mixture contains more of one enantiomer than the other, e.g. more +HupA than −HupA.

Some brain injuries such as penetrating traumatic brain injuries and blast induced traumatic brain injuries involve the EAA and NMDA receptor pathway which results in SE causing seizures, SE, neuropathogenesis and neuropathology. It was found that huperzine A compounds treat, prevent, reduce or inhibit seizures and neuropathology resulting from brain injuries which result in overstimulation of the NMDA receptor pathway. Therefore, the present invention provides methods for treating, preventing, reducing or inhibiting seizure, SE causing seizures, SE, neuropathogenesis and neuropathology caused by a brain injury which involves the EAA and NMDA receptor pathway comprising administering to a subject in need thereof a huperzine A compound, e.g. +HupA or a huperzine A compound as set forth in FIG. 12.

### +HupA and SE Causing Seizures and SE Induced by OP Compounds

To demonstrate the protective efficacy of +HupA against SE causing seizures and SE induced by OP compounds, such as soman and sarin, diisopropyl-n-fluorophosphate (DFP) and a rat radiotelemetry model were used. SE causing seizures were induced by subcutaneous (s.c.) administration of DFP, and the animals were treated with +HupA pre- or post-exposure. As provided below, +HupA was found to inhibit DFP induced SE causing seizure in rats.

Male Sprague-Dawley rats (200-250 g, Rattus norvegicus) were purchased from Charles River Laboratories (Wilmington, Mass.). The rats were housed individually in cages with a 12 hr light/dark cycle. Food and water were available ad libitum, and a one-week stabilization period preceded surgery and experimentation.

TL11M2-F40-EET bipotential radiotelemetry probes purchased from Data Sciences International (St. Paul, Minn.) were surgically implanted in the rats using methods known in the art. Specifically, each rat was anesthetized in a chamber with isoflurene gas (2-5% isoflurene, oxygen 1 L/min flow rate) and injected with buprenorphine (0.1 mg/kg i.m.) to alleviate the discomfort from the surgical procedure. In order to keep the rat anesthetized during surgery, the rat's nose and mouth were fixed at the end of a tube that pumped isoflurene gas (2-3% isoflurene, oxygen 1 L/min flow rate). Body temperature was maintained by a water-heated blanket. The rat's head was held in place by a stereotax (David Kopf Instruments, Tujunga, Calif.). The dorsal surfaces of the rat's abdomen and head were then shaved and cleaned. Two initial incisions were made: one along the midline of the back and 7.5 cm anterior to the tail, and one along the dorsal midline of the head. Two wires (negative, positive) were tunneled subcutaneously from the posterior incision to the anterior incision. Then the skull was then cleaned with gauze, and any open veins or arteries were closed by surgical cautery. Two 1 mm holes were drilled into which two screws were inserted. The screws were placed 3 mm anterior to the lambdoid suture and 3 mm on each side of the sagittal suture. The positive wire went to the right, posterior screw, and the negative wire to the left screw. Tissue safe adhesive was used to keep the wires separated and in place.

While the acrylic was drying, the positive ECG wire was subcutaneously tunneled along the left side of the rat's abdomen to the xiphoid process, and the negative ECG wire was subcutaneously tunneled along the right anterior side of the rat's abdomen to the right pectoral muscle. The probe was then inserted subcutaneously on the left dorsal side of the rat. The rat was sutured using Ethicon sutures (Piscataway, N.J.) and given seven days to recover.

The rats were randomly assigned to 5 experimental groups (n=6) and one control group (n=6). The rat's telemetry probes were activated, and the cage was centered on a telemetry receiver. Radiotelemetry data was monitored 30 min before injection, up to 24 hr post injection. All groups were injected with pyridostigmine bromide (PB) (0.026 mg/kg) at the beginning of the experiment 20-30 min prior to the DFP (4 mg/kg, s.c.) injection. A combination of atropine and 2-PAM (2 mg/kg and 25 mg/kg) was injected one min after the DFP injection in order to prevent immediate death. The saline control group received only i.p. injections of 100 μl PBS following PB injection. Experimental groups received either a 30 min pre-exposure treatment or a 1, 5, or 10 min post-exposure treatment of +HupA (3 mg/kg), while DFP control groups received 100 μl PBS as treatment. The number of rats that survived 24 hr was recorded. If a rat died from seizure before the end of the study, its brain was collected. After 24 hr, all surviving rats were euthanized. The brain, liver, lung, kidney, heart, spleen, and blood were collected from each rat.

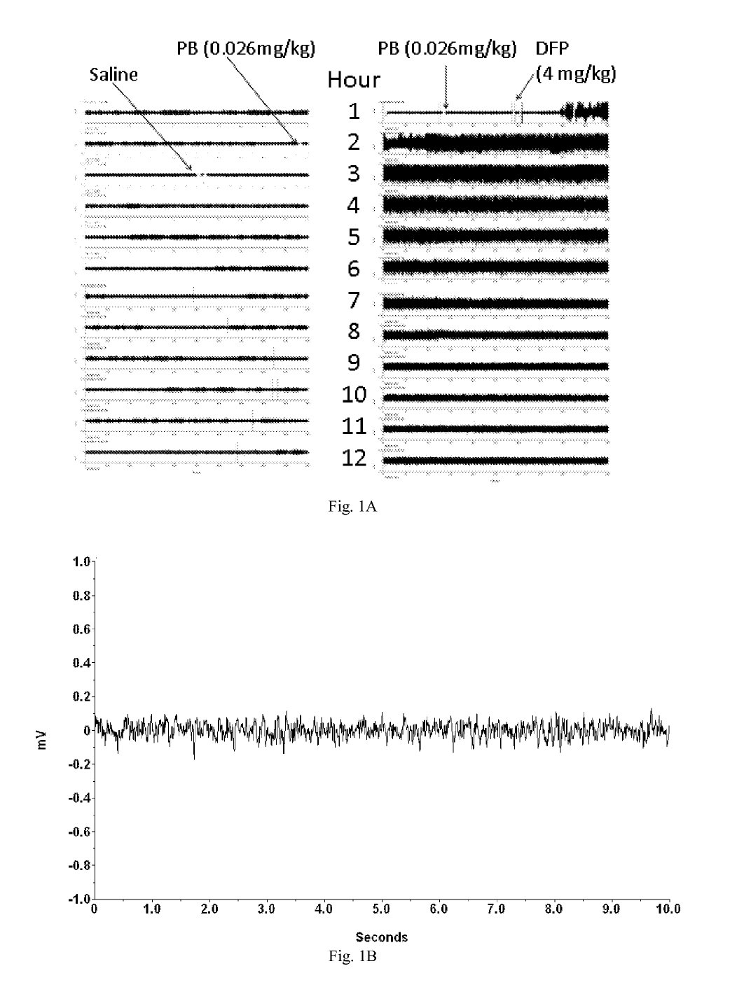

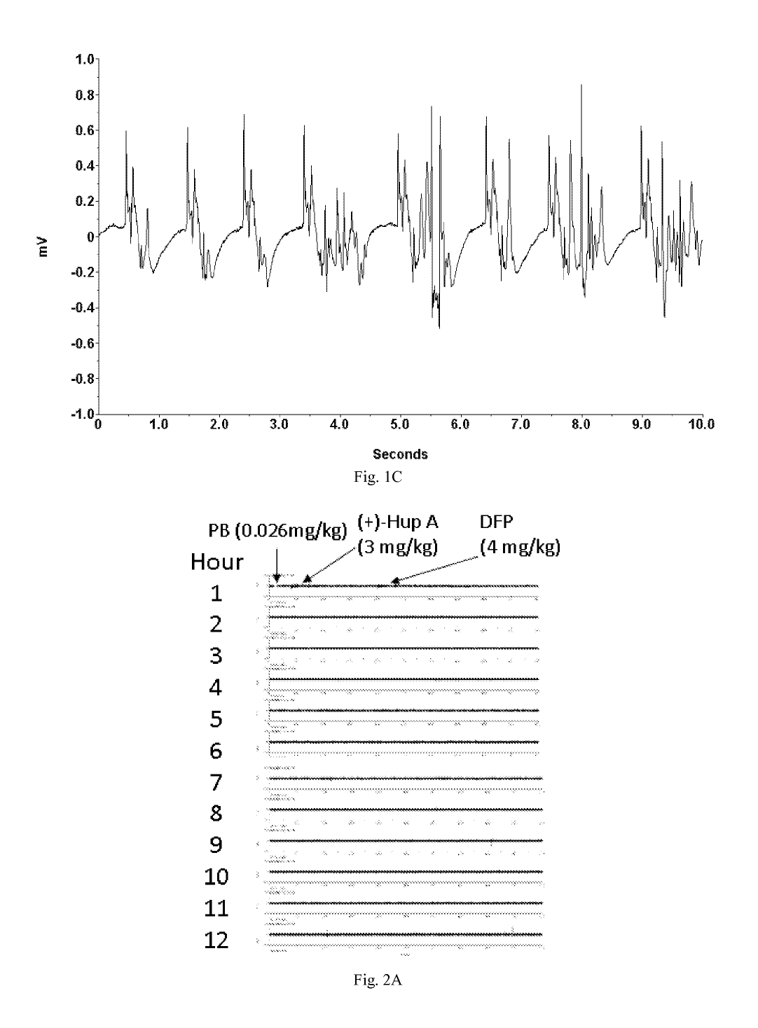

Saline Controls. EEG from rats administered PB (0.026 mg/kg) and saline (i.m.) showed normal brain activity, and showed no signs of seizures, SE causing seizures or SE. See FIG. 1A, Panel A. The survival of the animals following saline administration was 100% (n=6). A 10 second enlarged EEG recording from 3 hr after saline injection also shows that rats injected with saline did not experience any seizures, SE causing seizures or SE. See FIG. 1C. The EEG voltage ranged from +0.25 mV to −0.25 mV, and remained consistent throughout the 24-hr time period. The heart rates of saline control animals remained within the normal range of 300-500 bpm. Body temperature remained at an average of 36° C., also within normal limits. Physical activity, as calculated by the number of times the rat crossed the middle of the receiver, was consistent over the 24-hr period.

DFP Controls. EEG from rats administered DFP (4 mg/kg, s.c.) and saline (100 μl, i.m.) showed SE causing seizures which soon progressed to SE 12-15 min after DFP exposure. FIG. 1A, Panel B. A 10 sec enlarged EEG recording showed that rats injected with DFP continued to experience SE 3 hr after DFP exposure. See FIG. 1C. The EEG voltage initially ranged from +0.8 mV to −0.8 mV beginning 14 min after DFP injection. 6 hr after DFP injection, the EEG voltage dropped to an average range of +0.6 mV to −0.6 mV, but never reached within normal EEG voltage limits. The survival of the animals following DFP administration was 83.3% (n=6). The heart rates of DFP control animals dropped from an average of 500 bpm to an average of 350 bpm within 3.5 hr of DFP injection, and remained constant for the next 10 hr. The heart rate then dropped to an average of 325 bpm and remained constant until the end of the recording period. Body temperature dropped significantly 3.5 hr after DFP injection, reaching a low of 31° C. 8.5 hr after DFP injection. Body temperature then began to rise to an average of 33° C. for the remainder of the experiment. Physical activity significantly dropped 3.5 hr after DFP injection. There was a noticeable quiet period between 3.5 and 9 hr after DFP exposure, as well as between 14 and 17.5 hr after DFP exposure. A small amount of physical activity was then observed intermittently for the remainder of the experiment.

+HupA Pre-Treatment. EEG from rats administered +HupA (3 mg/kg, i.m.) followed by DFP (4 mg/kg, s.c.) 30 min later showed no signs of SE causing seizures or SE, as compared to DFP controls. See FIG. 2A. A 10 sec enlarged EEG recording showed that rats pre-treated with +HupA did not experience seizure activity at all 3 hr after DFP exposure, and looked much like the saline control. See FIG. 2B. The EEG voltage ranged from +0.1 mV to −0.1 mV throughout the entire radiotelemetry recording. The survival of the animals that received +HupA pre-treatment was 100% (n=6).

The heart rates of +HupA pre-treatment animals dropped from an average of 500 bpm to an average of 400 bpm within 3.5 hr of DFP injection, and ranged within normal levels for the remainder of the experiment. Body temperature dropped significantly about 3.5 hr after DFP injection, reaching a low of 32° C. 3.5 hr after DFP injection. Body temperature then began to rise to an average of 35° C. for the remainder of the experiment. The animals remained consistently active for the duration of the 24-hr experiment. There was no quiet period observed in pre-treated rats.

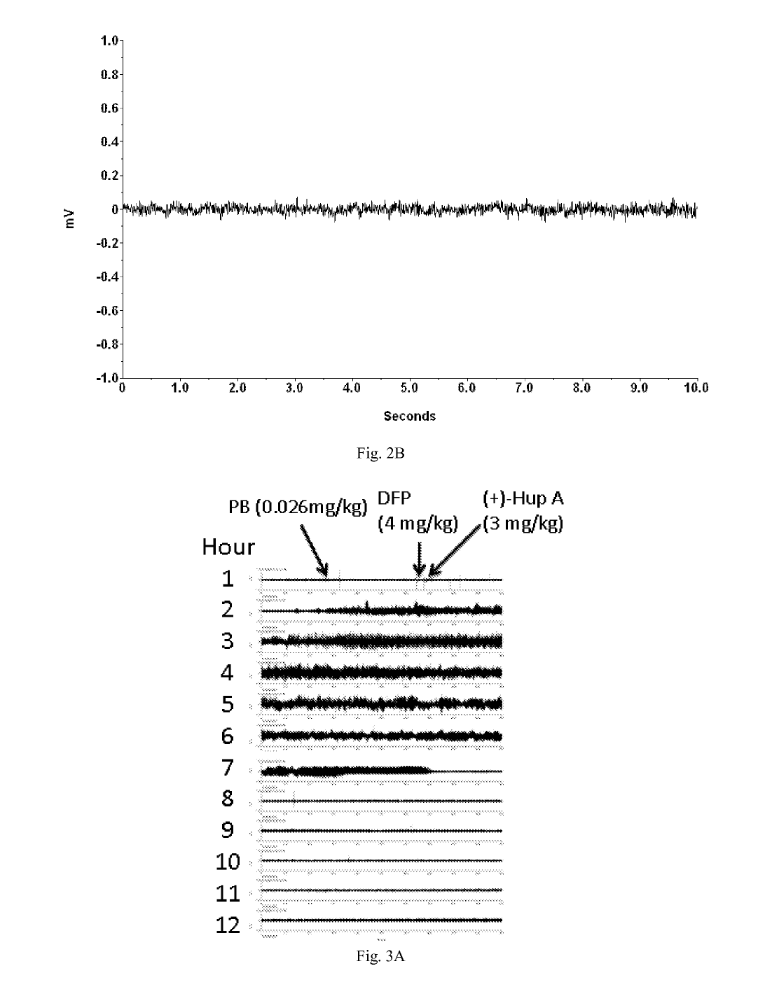

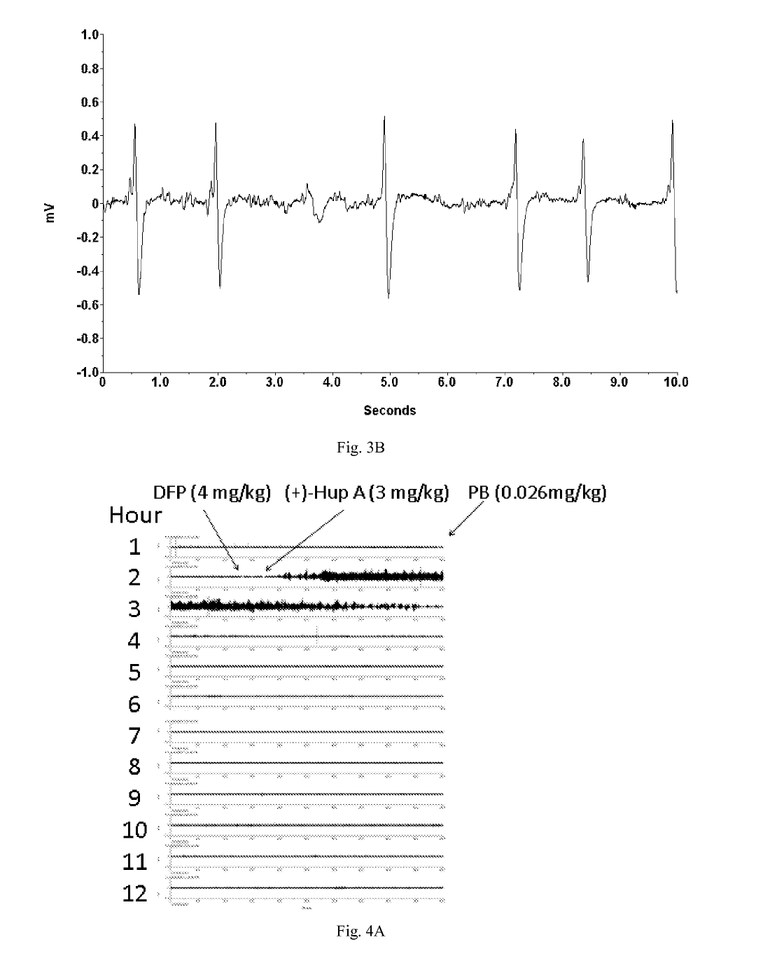

+HupA 1 Minute Post-Treatment. Rats were administered DFP (4 mg/kg, s.c.) followed by +HupA (3 mg/kg, i.m.). These animals show signs of SE causing seizures about 36 min after DFP injection which progressed to SE about 48 min after DFP injection, which then continued for the next 5 hr and 15 min. EEG readings within this timeframe ranged from +08 mV to −0.8 mV. The seizure then quickly decreased in intensity and remained between +0.25 mV to −0.25 mV for the remainder of the experiment. See FIG. 3A. A 10 sec enlarged EEG recording showed the rats treated with +HupA 1 min after DFP exposure still showed seizure activity 3 hr after DFP injection. See FIG. 3B. The survival of the animals that received 1-min post +HupA treatment was 87.5% (n=8). The heart rates of +HupA treated animals increased from an average of 400 bpm to an average of 450 bpm within 3 hr of DFP injection, and then dropped to an average of 425 bpm for the remainder of the experiment. Body temperature increased from 35° C. to 37° C. immediately after DFP exposure. Three hr after DFP injection, body temperature dropped from 37° C. to 33° C., then steadily rose back to an average of 35° C. Physical activity significantly decreased 2 hr after DFP exposure. A quiet period was observer between 2 and 4 hr after DFP injection, after which physical activity resumed for about 1 hr. After this, no physical activity was observed for the remainder of the experiment.

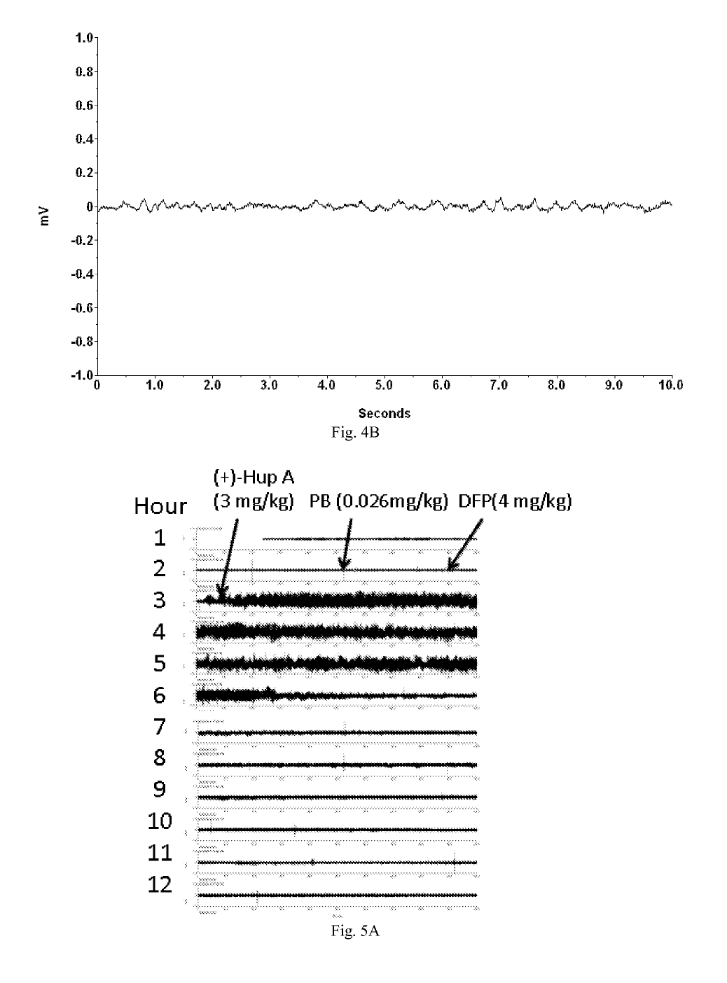

+HupA 5 Minute Post-Treatment. Rats were administered DFP (4 mg/kg, s.c.), followed by +HupA (3 mg/kg, i.m.) 5 min later. These animals showed signs of strong seizure activity about 8 min after DFP exposure, which continued for 45 min. EEG readings during this time ranged from +0.6 mV to −0.6 mV. The seizure activity then began to decrease in intensity over the next 45 min, with EEG readings ranging from an average of +0.4 mV to −0.4 mV. EEG readings then returned to normal levels for the remainder of the experiment, ranging from an average of +0.15 mV to −0.15 mV. See FIG. 4A. A 10 sec enlarged EEG recording showed that rats treated with +HupA 5 min after DFP exposure show no sign of SE causing seizures or SE 3 hr later, and are comparable to saline control animals. See FIG. 4B. The survival of the animals that received 5-min +HupA post-treatment was 96% (n=24). The heart rates of +HupA treated animals increased from an average of 450 bpm to an average of 500 bpm within 1 hr of DFP injection, and then dropped to an average of 400 bpm and remained constant for the remainder of the experiment. Body temperature dropped significantly about 3.5 hr after DFP injection, reaching a low of 31° C. 13 hr after DFP injection. Body temperature then began to rise and peaked at 35° C. at the end of the 24 hr recording. Physical activity dropped significantly 3.5 hr after DFP injection. There is a noticeable quiet period between 3.5 and 9 hr after DFP exposure, much like DFP control animals. However, unlike the DFP controls, physical activity resumed after 9 hr to normal levels until recording was completed.

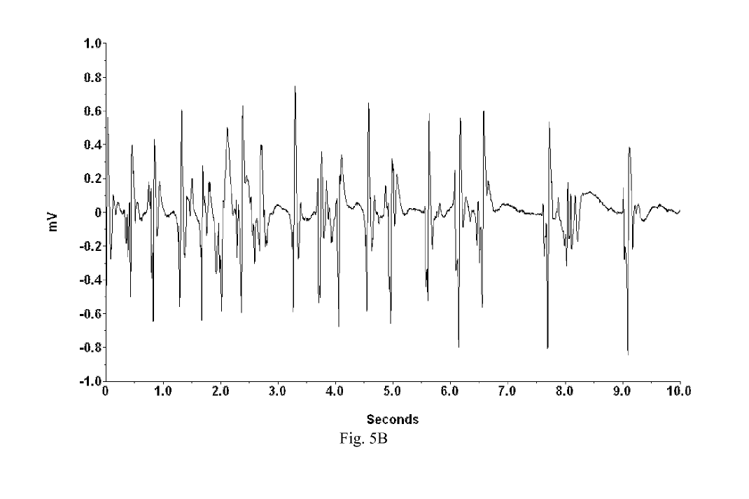

+HupA 10 Minute Post-Treatment. Rats were administered DFP (4 mg/kg, s.c.), followed by +HupA (3 mg/kg) 10 min later. These animals showed signs of strong seizure activity about 8 min after DFP exposure continuing for 3 hr, with EEG readings ranging from +0.8 mV to −0.8 mV. The seizure activity then began to decrease in intensity over the next 24 min, with EEG readings ranging from an average of +0.5 mV to −0.5 mV. Readings then decreased to an average of +0.3 mV to −0.3 mV and stayed there for the remainder of the experiment. See FIG. 5A. A 10 sec enlarged EEG recording showed that rats treated with +HupA 10 min after DFP exposure display signs of mild seizure, but not sustained SE. See FIG. 5B. The survival of the animals that received 10-min +HupA post-treatment was 83% (n=6). The heart rates of +HupA treated animals increased from an average of 400 bpm to an average of 500 bpm within 1 hr of DFP injection, and then dropped down to an average of 300 bpm 2 hr later. The animals' heart rate then dropped again to an average of 250 bpm 8 hr later, and remained constant for the remainder of the experiment. Body temperature initially increased from 35° C. to 37° C., then began to drop about 4 hr after DFP injection, reaching a low of 30.5° C. 17.5 hr after DFP injection and remained low for the remainder of the recording. Physical activity drops significantly 4 hr after DFP injection. There is a noticeable quiet period between 4 and 18 hr after DFP exposure. Physical activity was noticed occasionally until radiotelemetry recording was finished.

Blood AChE Activity. Using methods known in the art, blood AChE activities were assayed. AChE activity of rats treated with +HupA pre- and post-exposure showed significant increase in AChE activity in comparison to rats which received no treatment.

These results show that pre- and post-exposure treatment with +HupA protects against DFP-induced SE causing seizures and SE in the rat model. A single dose of +HupA provided 30 min before DFP injection was able to completely protect the rat from experiencing any SE causing seizures and SE for 24 hr. A single dose of +HupA provided 5 min after DFP injection was also able to completely eliminate any SE causing seizures and SE for the duration of the experiment and a single dose of +HupA provided 1 or 10 min after DFP exposure partially protected the rat from seizure activity. These experiments indicate that pre- and post-treatment with +HupA were effective in treating SE causing seizures and SE.

The AChE assay results show that although +HupA itself does not raise AChE activity, it allows the body to return to normal levels when treated at the most effective dosage and time. These experiments show that +HupA administered before and after exposure to an OP compound is able to significantly protect against SE causing seizures and SE caused by the OP compound.

Additional experiments showed that 1 min post-exposure treatment with +HupA also protect against 2LD₅₀ soman exposure by reducing seizures causing SE and neuropathology in a dose-dependent manner. Treatment with +HupA also reduced neuropathology in the hippocampus, CA1, CA3, Dentate gyrus, and Hylus regions. Huperzine A compounds 3, 4 and 90 as set forth in FIG. 12 were found to be protective against neuropathology resulting from exposure to DFP and exposure to soman.

Therefore, in some embodiments, the present invention provides methods for treating, preventing, inhibiting, or reducing an SE causing seizure, SE, neuropathogenesis, and/or a neuropathology caused by an OP compound which comprises administering to a subject in need thereof a therapeutically effective amount of a huperzine A compound, such as +HupA or one as set forth in FIG. 12. In some embodiments, the huperzine A compound is administered before, during, or after, or a combination thereof, exposure to the OP compound. In some embodiments, the huperzine A compound is administered before and after exposure to the OP compound. In some embodiments, the huperzine A compound is administered up to 30 min prior to OP exposure. In some embodiments, the huperzine A compound is administered after 1 min up to about 10 after OP exposure. In some embodiments, the huperzine A compound is administered as an enantiopure composition, e.g. +HupA. In some embodiments, the huperzine A compound is administered as a mixture of two or more different huperzine compounds. In some embodiments, the huperzine A compound, such as +HupA, is administered as a mixture with −HupA in order to additionally provide the protective benefits of the anti-cholinergic activity of −HupA. In some embodiments, the mixture is a racemic mixture. In some embodiments, the mixture contains more of one enantiomer than the other, e.g. more +HupA than −HupA.

### PRO-2-PAM

The current recommended treatment for exposure to OP compounds is administration of an anti-cholinergic compound, such as atropine sulfate, which antagonizes the effects of excess ACh at muscarinic receptor sites, and an oxime, such as 2-PAM, which reactivates any unaged, inhibited ChE and an anticonvulsant, such as benzodiazepine, to ameliorate seizures.

2-PAM is a quaternary oxime which is administered to subjects after exposure to an OP compound in order to reactivate any unaged ChE in the peripheral nervous system (PNS) as it is generally accepted by those skilled in the art that 2-PAM does not cross the blood brain barrier (BBB).

In 1978, Rump et al. showed that Pro-2-PAM reactivated brain AChE in amounts greater than 2-PAM. See Rump et al. (1978) Arch In Pharmacodyn 232:321-332. Clement, however, conducted studies which showed that although Pro-2-PAM crosses the BBB, 2-PAM provided superior protection against OP compounds and that there was a lack of correlation between reactivation of brain AChE and an increase in protective ratio. See Clement (1978) Suffield Technical Paper No. 487.

In 1980, Boskovic et al. found that Pro-2-PAM was less effective than 2-PAM against paraoxon poisoning. See Boskovic et al. (1980) Toxicol Appl Pharmacol 55:32-36.

In 1982, Kenley et al. conducted experiments showing that both 2-PAM and Pro-2-PAM do not reverse the behavior effects of DFP. See Kenley et al. (1982) Pharmacol Biochem & Behavior 17:1001-1008.

In 2003, Sakurada et al. found that about 10% of 2-PAM administered i.v. penetrates the BBB, which may effectively reactivate AChE in the brain. See Sakurada et al. (2003) Neurochem Research 28(9):1401-1407. Some speculate that the amounts of 2-PAM which cross the BBB are not therapeutically relevant amounts.

However, in 2008, Lorke et al. found that the protective effects against OP poisoning by 2-PAM is probably due to reactivating ChE in the PNS rather than the brain since intrathecal delivery of 2-PAM did not appear to provide better protection over i.m. injections of 2-PAM.

In 2000, Prokai et al. suggested that conversion of Pro-2-PAM to 2-PAM is analogous to the oxidation of nicotinamide adenine dinucleotide (NADH) to NAD, a coenzyme associated with several oxidoreductases and cellular respiration, which are intracellular reactions. See Prokai et al. (2000) Med Res Rev 20:367-416. This conversion mechanism indicates that the conversion would result in the biotransformation of Pro-2-PAM to 2-PAM intracellularly, which is consistent with observations that (1) 2-PAM is effective in the periphery as 2-PAM may reactivate extracellular AChE which can then act on the ACh in the neuro-muscular (PNS) junctions, and (2) Pro-2-PAM does not exhibit protection that is better than 2-PAM. Once intracellular, 2-PAM cannot escape and become extracellular. Thus, it was believed that Pro-2-PAM will not be effective in treating SE causing seizures and SE because the converted 2-PAM will be trapped inside the cells and can not act on extracellular AChE in the brain.

Because of these experiments and knowledge in the art it was questionable whether Pro-2-PAM and 2-PAM would provide any protective benefits against seizures, SE causing seizures and SE induced by OP exposure, other than some reactivation of AChE in the brain and peripheral blood. Therefore, further experiments were conducted in order to determine if 2-PAM, Pro-2-PAM, or both can be used to effectively treat seizures, SE causing seizures and prevent SE caused by exposure to OP compounds. The effects of Pro-2-PAM after OP exposure were documented and correlated using (a) surgically implanted radiotelemetry probes that recorded electrocardiogram (ECG), electroencephalogram (EEG), body temperature, and physical activity, (b) histopathology analysis of brain, and (c) cholinesterase activities in the PNS and CNS. Guinea pigs were used as the model for OP poisoning because its repertoire of OP detoxifying enzymes matches the human enzyme complement. The guinea pig brains in this study were processed for histopathology from 2 mm coronal sections, and stained with either hematoxylin and eosin (H&E) or fluoro-jade to determine neuropathogenesis caused by OP exposure.

As disclosed below, 2-PAM was found to be ineffective at reducing seizures, SE causing seizures and SE in DFP-exposed animals. In contrast, Pro-2-PAM significantly suppressed and then eliminated seizure activity and since Pro-2-PAM inhibited seizure activity, progression to SE was prevented. Specifically, two month old adult male guinea pigs were fasted for several hours, anesthetized (2-5% isoflurane, oxygen 1.5 L/min), shaved on the head and back, and their heads placed in a stereotaxic instrument (David Kopf Instruments, Tujunga, Calif.). The radiotelemetry system consisted of 8 receivers and TL11M2-F40-EET bipotential radiotelemetry probes (DSI, St. Paul, Minn.) turned on and off by hand with a magnet. Probes were reused and sterilized using 4% glutaraldehyde and handled as instructed by the manufacturer. Briefly, the surgery proceeded as follows: radiotelemetry probes were surgically implanted under the back skin, with wire leads fixed to the skull, chest muscle, and abdominal muscle to record brain activity (EEG), heart rate (ECG), and body temperature, respectively. See Tetz et al. (2006) Toxicol Ind Health 33(6):255-266. Cyano-acrylate glue was used to keep the skull electrodes in place. Incisions were sutured using Ethicon sutures (Piscatawy, N.J.) and covered with TISSUEMEND glue (Webster's Veterinary Supply, Sterling, Mass.). The guinea pigs were housed individually in microisolator cages with a 12 hr light/dark cycle. Food and water were available ad libitum, and a one week stabilization period preceded surgery and experimentation.

On the day of the experiment, the standard military exposure paradigm was used (Newmark (2004) Arch. Neurol. 61(5):649-652) and only the oxime delivery time was modified as follows: guinea pigs were pretreated (i.p.) with PB at 0.026 mg/kg. PB is a reversible inhibitor of AChE activity, but does not cross the BBB and therefore does not sequester CNS ChEs. After 20 min, the animals were injected (s.c.) with DFP (8 mg/kg) followed 1 min later by atropine methyl bromide (i.m., 2 mg/kg, which does not penetrate the CNS). At various times post-OP exposure, equivalent doses of 2-PAM or Pro-2-PAM were injected i.m. (1.5 auto-injector, 13 mg/kg) to approximate the use of the Mark I nerve agent antidote kit provided to military personnel. The EEG, ECG, body temperature, and activity of the animals were continuously monitored and telemetry recorded for 24 hr. Guinea pigs were euthanized after 24 hr (in some studies 1.5 hr) by injecting 75 mg/kg pentobarbital followed by terminal cardiac puncture exsanguination. Brain, blood, and diaphragm tissues were frozen on dry-ice for ChE assays. Some whole brains, taken from heparinized saline perfused animals, were thawed and dissected into eight distinct brain regions for AChE assays: frontal cortex, rear cortex, hippocampus, thalamus, hypothalamus, midbrain, cerebellum, and brain stem.

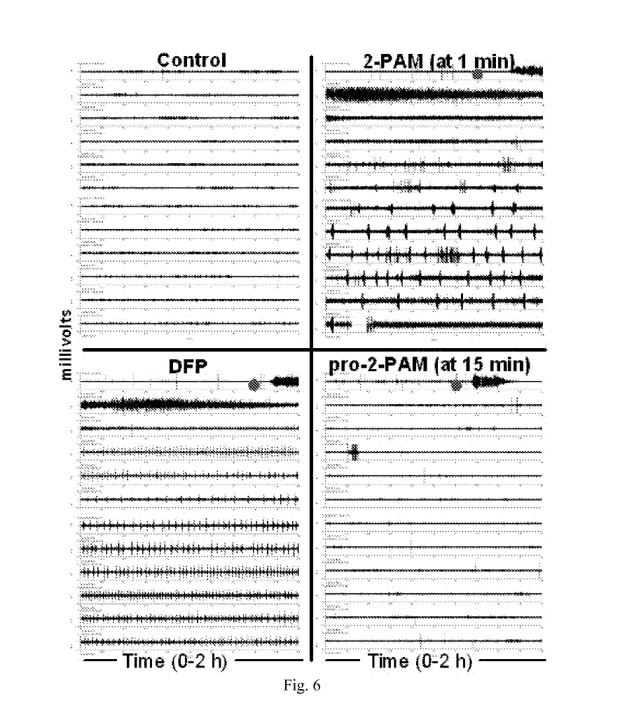

Control, DFP alone, or DFP followed by 2-PAM or Pro-2-PAM treated animals were continuously monitored for 24 hr for brain activity (EEG, FIG. 6) and heart rate (ECG), body temperature, and physical activity. These parameters allowed the protective effects of Pro-2-PAM to be evaluated in comparison to 2-PAM after OP (DFP) exposure. The amount of oxime (2-PAM or Pro-2-PAM) administered as a single dose was equivalent to the 1.5 human auto-injector dose by body weight, which was found to be most efficacious, although doses of 1, 2, and 3 auto-injector equivalents were also evaluated (not shown). Control animals received PB, methyl atropine bromide, and saline instead of DFP and/or oximes.

Control guinea pigs displayed EEG tracing with only minor single spikes due to instrumentation noise. See FIG. 6, Control. In contrast, guinea pigs exposed to DFP produced intense SE causing seizures that continued for the full 24 hr, if they survived. See FIG. 6, DFP. 2-PAM was ineffective at reducing SE causing seizures and SE in DFP-exposed animals, since the guinea pigs exhibited seizure activity for the full 24 hr recording period. See FIG. 6, 2-PAM. 2-PAM was rapidly injected at 1 min post-DFP exposure. In notable contrast, Pro-2-PAM prevented seizure activity in the standard military regimen of dosage and time, 1 min after OP exposure (not shown). Remarkably, Pro-2-PAM abrogated seizure activity even when injected 15 min later. See FIG. 6, Pro-2-PAM. These results show that Pro-2-PAM, but not 2-PAM, effectively treats, prevents, inhibits or reduces seizures, SE causing seizures and SE caused by exposure to OP compounds.

Therefore, in some embodiments, the present invention provides methods for treating, preventing, inhibiting, or reducing seizures, SE causing seizures and SE caused by an OP compound which comprises administering to a subject in need thereof a therapeutically effective amount of Pro-2-PAM. In some embodiments, Pro-2-PAM is administered before, during, or after, or a combination thereof, exposure to the OP compound. In some embodiments, Pro-2-PAM is administered in combination with 2-PAM or another oxime in order to provide additional PNS protective benefits.

Neuropathology. Twenty four hr post-exposure, guinea pigs were euthanized as above, the brain removed, and forebrain taken for cholinesterase activity assays. The remainder of the brain was subjected to immersion fixation, for at least several weeks, in 4% formaldehyde (stabilized with 0.5% methanol). Next, the formaldehyde-preserved guinea pig brains were transverse sectioned using a rodent brain matrix (model: RMB-5000C; ASI Instruments, Inc., Warren, Mich.). Two sequential transverse sections, “A” and “B”, of 2 mm thickness were cut from each brain, using microtome blades hand dropped into the matrix. Section “A” was cut starting at the nose of hippocampus and section “B” was cut starting near the back of the hippocampus, adjacent to the midbrain. Both sections were processed into microscope slides containing paraffin embedded 6 μm transverse sections (microtome cut) stained with H&E or fluoro-jade in duplicate (FD Neurotechnologies, Inc; Ellicott City, Md.). H&E stain is reactive towards membrane lipids and proteins, and highlights the general structural morphology of all cells. In contrast, fluoro-jade stain penetrates only leaky membranes and thus highlights dead cells. Prepared slides were examined at 40× magnification under an Olympus axial light microscope equipped with an image capture camera (Olympus Provis AX80/DP70; Olympus, Center Valley, Pa.). Standard bright field and fluorescence (FITC filter) illuminations were used on the H&E and fluoro-jade stained slides, respectively. The middle lobe of the piriform cortex, a distinct brain region known to be sensitive to OP nerve-agent induced damage and a site of seizure initiation/propagation, was examined in the section “A” slides using methods known in the art. See Carpentier et al. (2000) Neurotoxicol 21(4):521-540. Likewise, in the section “B” slides, the lower-outside pyramidal layer of the hippocampus (CA1-CA2 region) was chosen for examination. Photographic images were captured of neurons and granular cells comprising the selected regional zones in both sections.

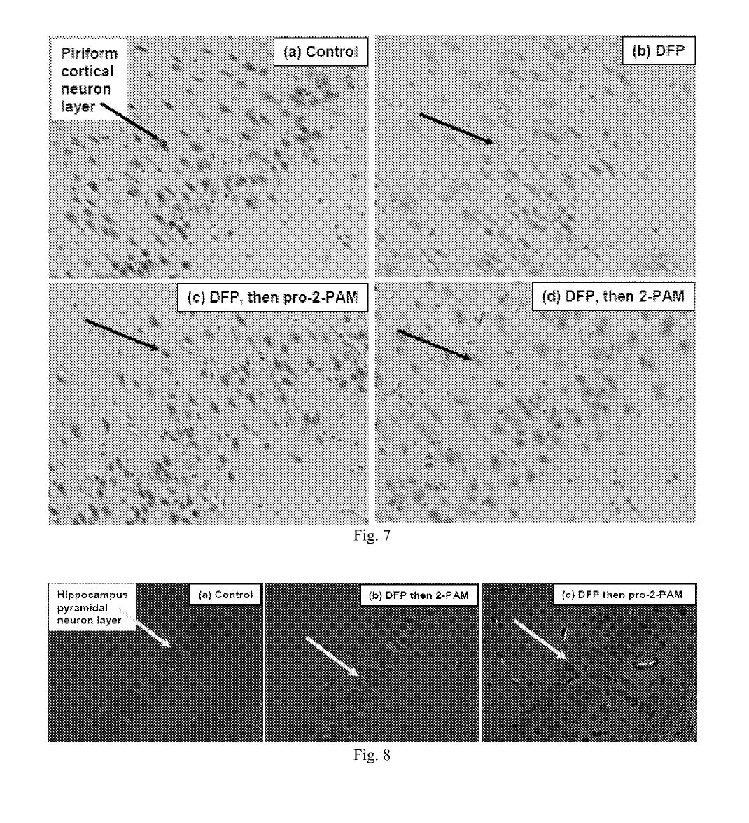

Using microscopy, distinct differences between 2-PAM or Pro-2-PAM treatments were noted in “A” and “B” sections at the cellular level magnification of 40×, where “A” surveys the piriform cortical neuron layer and “B” the hippocampal pyramidal neuron layer. Under H&E stain, as shown in FIG. 7, Panels b and d, which represents a section “A” slice of the brain of DFP or DFP then 2-PAM treated guinea pigs, respectively, the neurons in the piriform cortex showed markedly swollen morphology along with deformed nuclei. These observations contrast with the brain slice from control (PB+atropine, only) and DFP then Pro-2-PAM treated animals (FIG. 7, Panels a and c, respectively), where the piriform cortex neurons lack the signs of neuronal necrosis or degeneration seen with 2-PAM treated animals. See FIG. 7, Panel d.

After fluoro-jade stain, for section “B” slices, the lower-outside pyramidal layer of the hippocampus exhibited heavily distorted and missing granular cells and neurons in the DFP then 2-PAM treated animals (FIG. 8, Panel b), and dead cells as visualized by the highlighted fluorescent staining were found interspersed and disrupting the order of the pyramidal layer. In contrast, Control (PB+atropine only, FIG. 8, Panel a) hippocampus pyramidal neuron layer was well defined. The DFP then Pro-2-PAM layer from treated animals, however, displayed fewer signs of OP toxicity with only swollen cells in this region. Also, the DFP then Pro-2-PAM treated animals lacked dead cells as defined by fluoro-jade staining (FIG. 8, Panel c). Slices from control receiving either oxime but no DFP showed no discernable cellular changes in the brain regions examined for sections “A” and “B” (histopathology images not shown).

These experiments demonstrate that Pro-2-PAM, but not 2-PAM, is effective in treating seizures, SE causing seizures, SE, neuropathogenesis, and neuropathology caused by exposure to OP compounds despite evidence in the art that: (1) there is a lack of correlation between reactivation of brain AChE and an increase in protective ratio; (2) Pro-2-PAM was less effective than 2-PAM against paraoxon poisoning; (3) 2-PAM and Pro-2-PAM do not reverse the behavior effects of DFP; and (4) the protective effects against OP poisoning by 2-PAM is due to reactivating ChE in the PNS.

Therefore, in some embodiments, the present invention provides methods for treating, preventing, inhibiting, or reducing seizures, SE causing seizures, SE, neuropathogenesis, and neuropathology (including neurotoxicity, neuronal necrosis and neuronal degeneration) caused by exposure to an OP compound which comprises administering to a subject in need thereof a therapeutically effective amount of Pro-2-PAM. In some embodiments, Pro-2-PAM is administered before, during, or after, or a combination thereof, exposure to the OP compound. In some embodiments, Pro-2-PAM is administered during or after exposure to the OP compound. In some embodiments, Pro-2-PAM is administered in combination with 2-PAM or another oxime, in order to provide additional PNS protective benefits.

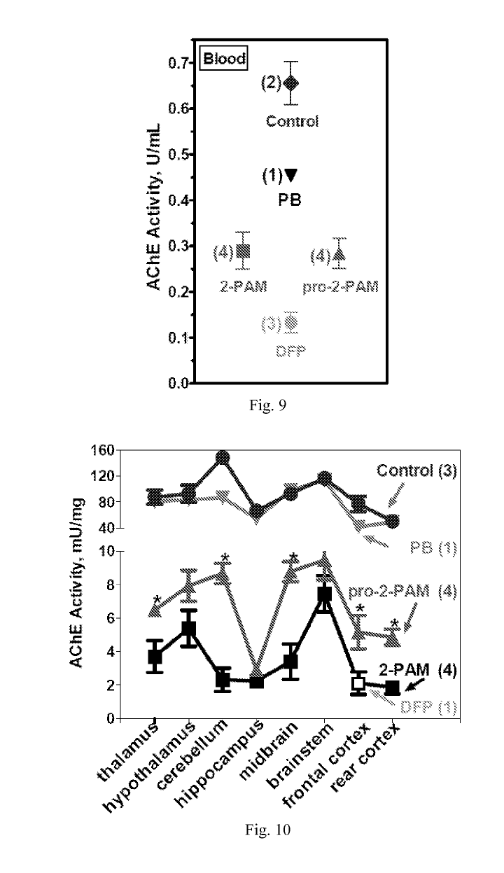

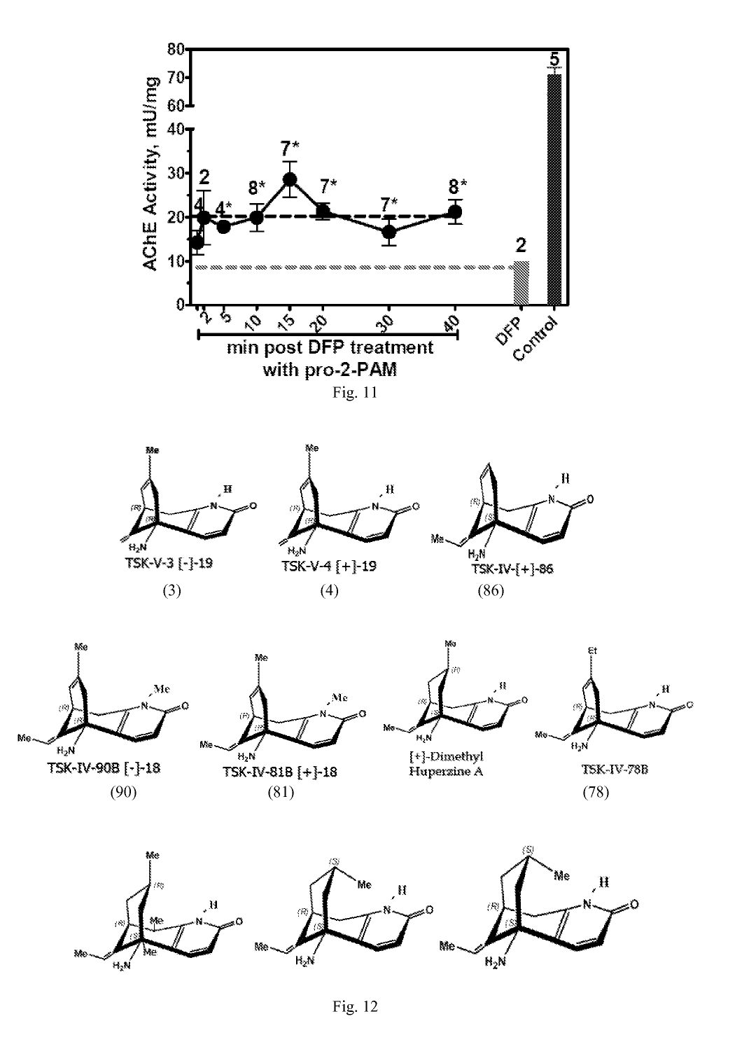

AChE Activity Assay. Blood and brain AChE activities were assayed using methods known in the art. It was found that AChE activities in blood and diaphragm from animals treated 2-PAM and Pro-2-PAM were similar. See FIG. 9. Both 2-PAM and Pro-2-PAM resulted in brain AChE reactivation when administered i.m. after OP exposure. However, distinct regional areas of the brains showed AChE activity at 1.5 hr after OP exposure in Pro-2-PAM treated animals that were higher than that of 2-PAM treated animals. In particular, six of eight distinct regional areas of brain showed significantly higher (p≦0.05) AChE activity, at least about 2-fold, at 1.5 hr after DFP exposure in Pro-2-PAM treated animals compared to 2-PAM treated animals. See FIG. 10. The data in FIG. 10 demonstrates that 2-PAM did not pass the BBB at therapeutically relevant doses or reactivate frontal cortex AChE since DFP and 2-PAM treated animals showed the same inhibited AChE activity. FIG. 11 shows the AChE activity of brain frontal cortex at 24 hr with Pro-2-PAM therapy between 1 and 40 min post-DFP exposure. There was greater than a 2-fold average increase in AChE activity (upper black dashed line) compared to DFP only treated animals (lower gray dashed line) (p≦0.05). Thus, Pro-2-PAM partially restores CNS AChE.

Therefore, the present invention is directed to methods of treating, reducing or inhibiting seizures, SE causing seizures, SE and neuropathogenesis caused by exposure to an OP compound which comprise administering Pro-2-PAM in a therapeutically effective dose to a subject in need thereof. In some embodiments, Pro-2-PAM is administered prior to the time the OP compound ages the ChE. For example, since sarin ages AChE in more than 1 hr, Pro-2-PAM may then be administered up to about 1 hr after exposure to sarin. However, soman ages AChE in about 2 min (T_{1/2}). Thus, Pro-2-PAM is preferably administered within about 2 min of exposure to soman.

Pro-2-PAM Conversion. After discovering that Pro-2-PAM is effective against SE causing seizures and SE, the conversion mechanism was investigated as the effectiveness of Pro-2-PAM is inconsistent with the NAD/P intracellular conversion mechanism proposed by Prokai et al. Specifically, the biotransformation of Pro-2-PAM proceeds to completion within minutes at physiological doses of riboflavin, in contrast to the NAD/P intracellular conversion which was incomplete after 30 min and therefore would fail to produce sufficient 2-PAM at relevant time frames to reactivate AChE, i.e. before AChE aging. In addition, the NAD/P intracellular conversion would counterproductively sequester 2-PAM within cells, thereby precluding its reactivation of extracellular AChE at neuronal junctions. Unexpectedly, it was discovered that once lipophilic Pro-2-PAM passes through the BBB, it is converted extracellularly to 2-PAM preferentially by riboflavin and flavin adenine dinucleotide (FAD). Thus, in some embodiments, the present invention is directed to providing 2-PAM extracellularly to the neuron-neuron synapse in the brain of a subject by administering Pro-2-PAM to the subject.

### Additional Pro-2-PAM Experiments

Additional experiments showed that 1 min post-exposure treatment with Pro-2-PAM also protected against multiple LD₅₀ of cutaneous soman exposure. In the above guinea pig skin model, OP compounds penetrate across the skin and then enter the systemic circulation Animals were weighed and their lateral sides clipped using an electric clipper with a #40 blade one day before the experiment. On the day of the study, animals had a central 3×4 cm area marked using a permanent marker as the site for OP application, i.m. pyridostigmine (0.026 mg/kg) 30 min prior to OP exposure and then an i.m. injection (in a rear leg) using the combination of ketamine (32 mg/kg) and xylazine (4 mg/kg) 5 min prior to OP exposure. Guinea pigs in individual cages were placed in the chemical fume hood, and the OP compound was applied in a droplet, to the center of the marked area. Typical soman volume varied between 0.05 and 500 μl. Exactly 1 min post-exposure, guinea pigs were given atropine sulfate (16 mg/kg) in one leg i.m. and in the other 2-PAM or Pro-2-PAM in the other leg Animals were observed continuously for the first 4 hr after exposure and intermittently for the next 4 hr or to the end of the work day. Signs of soman intoxication and the time of onset of each were recorded Animals were evaluated at 24 hr post-exposure, and surviving animals were euthanized by injecting 75 mg/kg pentobarbital followed by terminal cardiac puncture exsanguination. Brain, blood, and diaphragm tissues were frozen on dry-ice for ChE assays. In this guinea pig model, the LD₅₀ for soman with saline (control) treatment yielded 11.3 mg/kg, while 3 autoinjector equivalents of 2-PAM (25.7 mg/kg) increased the LD₅₀ to 66.7 mg/kg soman. This indicates the PR (protective ratio) of 2-PAM is about 5.9, a significant improvement in survivability of the animals. Treatment with equivalent Pro-2-PAM provided an LD₅₀ of 119 mg/kg soman, and a PR of about 10.5, which is almost twice (i.e. 1.8×) that of 2-PAM. All survivability curves were sigmoidal in shape. Thus, Pro-2-PAM exhibited better protection than 2-PAM (10.5 vs 5.9, respectively). Additionally, H&E stained images of the hippocampal neuron layer of soman exposed (45 mg/kg) and 2-PAM treated animals exhibited marked disruption of this layer, swollen neurons, and indistinct nuclei, in contrast to soman exposed (91 mg/kg) and Pro-2-PAM treated animals which exhibited an intact hippocampal layer. Unexpectedly, in contrast to that observed in the art, demonstrate: (1) the importance of a stable composition of Pro-2-PAM, (2) neuroprotection, and (3) rapid biotransformation to 2-PAM in the CNS and increase survivability against cutaneous OP exposure.

Therefore, the present invention provides methods for increasing the survivability of a subject after cutaneous exposure to an OP compound, which comprises administering the subject Pro-2-PAM in a therapeutically effective amount.

### +HUPA and PRO-2-PAM

As disclosed herein, the beneficial effects of +HupA in treating OP induced SE causing seizures, SE and neuropathogenesis were discovered to be due to its NMDA antagonist activity in the EAA excitatory pathway after OP exposure rather than its activity as a ChE inhibitor. Also, as disclosed herein, it was discovered that Pro-2-PAM was effective in treating OP induced SE causing seizures, SE and neuropathogenesis, and that its protective mechanism is likely due to its ability to reactivate AChE in brain and reduce the buildup of ACh.

Because +HupA and Pro-2-PAM exhibit different protective mechanisms against seizures, SE causing seizures and SE caused by exposure to OP compounds, in some embodiments, the present invention is directed to combination treatments and compositions comprising both +HupA and Pro-2-PAM. Therefore, in some embodiments, the present invention provides methods for treating, preventing, inhibiting, or reducing seizures, SE causing seizures, SE and/or neuropathogenesis caused by exposure to an OP compound which comprises administering to a subject in need thereof a therapeutically effective amount of Pro-2-PAM and a therapeutically effective amount of +HupA. In these embodiments, Pro-2-PAM and +HupA may be administered at the same or different times before, during, or after OP exposure or a combination thereof. For example, +HupA without Pro-2-PAM may be administered prior to exposure to an OP compound, then after exposure, both Pro-2-PAM and +HupA may be administered at the same time.

### Other Therapeutic Observations

+HupA. It was found that animals exposed to NMDA after pre-treatment with 3 mg/kg +HupA showed normal physical activity and that the quiet period observed following NMDA exposure was completely eliminated in +HupA treated animals. Behavior of animals pre-treated with 3 mg/kg +HupA was very similar to normal rats by visual observation. +HupA pre-treatment also maintained a normal heart rate of about 300-500 bpm. The body temperature of rats pre-treated with +HupA tended to be very unstable, but remained within the normal temperature range throughout the 24 hr monitoring period. The physical activity of animals was not affected by post-NMDA exposure treatment with 3 mg/kg +HupA. It was also discovered that +HupA is devoid of the side-effects such as behavior decrements which are normally associated with NMDA ion-channel antagonists. Rats treated with post-exposure +HupA showed normal baseline body temperature throughout the 24 hr recording period. These experiments show that +HupA does not have any observable cardiovascular toxicity. Therefore, +HupA may be administered to subjects with littler or no observable cardiovascular toxicity.

Administration of Pro-2-PAM exhibited additional therapeutic advantages. For instance, the parameters of heart rate (BPM), body temperature (T, ° C.), and physical activity (counts/min) were recorded for 24 hr exposure to OP compounds results in prolonged hypothermia, bradycardia, and decreased activity due to fasciculation and fatigue, all of which remained depressed for at least 24 hr after exposure. See Gordon et al. (1996) Pharmacol Biochem Behav 55(2):185-94. Treatment with 2-PAM partially modulated these responses, e.g. a long lag phase was observed before body temperature returned to normal. However, Pro-2-PAM treatment abrogated DFP induced hypothermia and bradycardia and restored activity. Therefore, the present invention also provides methods of treating, preventing, inhibiting or reducing hypothermia and bradycardia and reduced activity caused by exposure to an OP compound.

### Delivery Methods

Pro-2-PAM and a huperzine A compound, e.g. +HupA, may be administered to a subject using methods, formulations and devices (such as autoinjectors, transdermal patches, and inhalers) known in the art which are compatible with Pro-2-PAM and/or the huperzine compound, such as +HupA.

Since Pro-2-PAM is relatively unstable in solution for an extended period, in some embodiments, Pro-2-PAM is maintained in its solid powder form just prior to use. In these embodiments, an autoinjector having a first compartment for storing the solid powder and a second compartment for storing the liquid solvent in which Pro-2-PAM is dissolved in just prior to injection may be used. See e.g. Clair et al. (2000) Eur J Pharm Sci 9:259-263. In embodiments where Pro-2-PAM is to be administered at the same time as the huperzine A compound, such as +HupA, the liquid solvent in which Pro-2-PAM is to be dissolved may comprise the huperzine A compound. Since Pro-2-PAM is readily dissolved in a slightly acidic solution, e.g. 0.9% sodium chloride, pH 5, and +HupA is also soluble in acidic solution, in some embodiments, the liquid solvent is an acidic solution.

In order to administer Pro-2-PAM prior to cholinesterase aging by an OP compound, the mixing time for dissolving Pro-2-PAM in solution is preferably less than about 30 sec. Thus, in some embodiments, the device used to deliver Pro-2-PAM comprises a mixer which rapidly mixes the Pro-2-PAM into solution just prior to delivery, i.e. as the injection mechanism is triggered.

In some embodiments, Pro-2-PAM and/or the huperzine A compound are, alone or in combination, microencapsulated or delivered in a liposome.

### Additional Combination Therapies

The methods and compositions of the present invention may further comprise at least one supplementary active compound. Suitable supplementary active compounds, which are known in the art, include anticholingerics, anticonvulsants, carbamates, benzodiazepines, antiepileptics, barbituates, anesthetics, oximes, and prodrug forms thereof. As used herein, a “prodrug” refers to compound that, when administered to a subject, is converted in vivo into a compound that is active or significantly more active than the prodrug itself.

As used herein the term “anticholinergic” means any chemical, drug or drug effect that causes partial or total blockage of the action of the neurotransmitter acetylcholine. Examples include anisotropine, atropine, belladonna, clinidiun, dicyclomine, glycopyrrolate, homatropine, hyoscyamine, mepenzolate, methantheline, methscopolamine, pirenzepine, propantheline, hyoscine, aprophen, azaprophen, benactyzine, biperiden, procyclidine, and the like.

Examples of anticonvulsants include acetazolamide, carbamazepine, clobazam, clonazepam, diazepam, divalproex sodium, ethosuximide, ethotoin, felbamate, fosphenyloin, gabapentin, lamotrigine, levetiracetam, mephenyloin, metharbital, methsuximide, methazolamide, oxcarbazepine, phenobarbital, phenyloin, phensuximide, pregabalin, primidone, sodium valproate, stiripentol, tiagabine, topiramate, trimethadione, valproic acid, vigabatrin, zonisamide, avizafone, dihydrodiazepam, midazolam, and the like.

As used herein the term “carbamate” refers to derivatives of carbamic acid, including salts and esters, including urethanes (ethyl esters of carbamic acid). Examples include rivastigmine; neostigmine; pyridostigmine; physostigmine; thiaphysovenine; phenserine; norphysostigmine; physostigmine salicylate, Aricept®, donepezil, galanthamine, or the like.

As used herein, a “benzodiazepine” is a compound having a core chemical structure that comprises a benzene ring fused to a diazepine ring. Examples include chlordiazepoxide, diazepam, midazolam, imidazenil, avizafone, dihydrodiazepam, midazolam, and the like.

As used herein, a barbiturate is a compound that acts as a CNS depressant. Examples include allobarbital, amobarbital, aprobarbital, alphenal, barbital, brallobarbital, phenobarbital, and the like.

Suitable anesthetics include procaine, amethocaine, cocaine, lidocaine, prilocaine, bupivacaine, levobupivacaine, ropivacaine, mepivacaine, dibucaine, desflurane, enflurane, halothane, isoflurane, methoxyflurane, nitrous oxide, sevoflurane, and the like.

Suitable oximes include 2-PAM, Pro-2-PAM, obidoxime, methoxime, HI-6, HLo-7, TMB-4, monoisonitrosoacetone, diacetylmonoxime, MMB-4, those set forth in U.S. Pat. No. 3,962,447, bis-oximes such as those set forth in Hammond et al. (2008) J Pharmacol Exp Ther 307(1):190-196, Pang Y—P et al. (2003) Chem Biol 10:491-502, and the like.

### Dosages and Kits

Pro-2-PAM, a huperzine A compound, e.g. +HupA, or both may be provided in a kit as a single dose or as multiple doses, alone or in combination with one or more doses of at least one supplementary compound. In some embodiments, a single dose is a therapeutically effective amount. Determination of a therapeutically effective amount and timing of administration of a given compound is well within the capabilities of those skilled in the art, especially in light of the detailed disclosure herein. The amounts given below are a guideline and those skilled in the art may optionally titrate doses or use graded doses of an agent to achieve desired activity and minimize side effects in a treated subject.

A therapeutically effective amount of 2-PAM ranges from about 1 to about 30 mg/kg, preferably about 8 to about 26 mg/kg, more preferably about 8.6 to 25.7 mg/kg. Typically, dosages rage from 0.2 mg/kg/day to 30 mg/kg/day.

A therapeutically effective amount of atropine is about 0.03 to 20 mg/kg, preferably about 0.03 to about 16 mg/kg. Typically, dosages rage from 0.2 mg/kg/day to 20 mg/kg/day.

A therapeutically effective amount of Pro-2-PAM ranges from about 1 to 40 mg/kg, preferably about 8 to about 34 mg/kg, more preferably about 11 to 34 mg/kg, most preferably about 17 mg/kg. Typically, dosages rage from 0.2 mg/kg/day to 40 mg/kg/day.

A therapeutically effective amount of a huperzine compound ranges from about 0.2 mg/kg to 100 mg/kg, preferably about 1 mg/kg to about 52 mg/kg. Typically, dosages rage from 0.2 mg/kg/day to 100 mg/kg/day.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

1. A method of treating, preventing, inhibiting, or reducing a seizure, status epilepticus, neuropathogenesis, or a neuropathology caused by exposure to an organophosphate compound in a subject in need thereof which comprises
administering to the subject Pro-2-PAM, a huperzine compound, or both.

2. The method of claim 1, wherein Pro-2-PAM or the huperzine compound is administered before, during or after exposure to the organophosphate compound.

3. The method of claim 1, wherein Pro-2-PAM and the huperzine compound are administered at the same time, different times, or both.

4. The method of claim 1, wherein the huperzine compound is administered as an enantiopure composition or as a mixture.

5. The method of claim 1, and further comprising administering 2-PAM, a second huperzine compound, or both.

6. The method of claim 5, wherein the second huperzine compound is −HupA.

7. The method of claim 1, which further comprises
administering at least one supplementary active compound selected from the group consisting of anticholingerics, anticonvulsants, carbamates, benzodiazepines, antiepileptics, barbituates, anesthetics, and oximes.

8. The method of claim 1, wherein the seizure is a SE causing seizure.

9. A kit which comprises Pro-2-PAM and the huperzine compound packaged together.

10. The kit of claim 9, further comprising at least one device for delivering Pro-2-PAM, the huperzine compound, or both to a subject.

11. The kit of claim 10, wherein the device is an autoinjector.

12. The kit of claim 11, wherein the autoinjector comprises a first compartment containing Pro-2-PAM and a second compartment containing the huperzine compound.

13. The kit according to claim 9, wherein Pro-2-PAM, the huperzine compound, or both are provided as a single dose or multiple doses.

14. The kit according to claim 9, wherein Pro-2-PAM and the huperzine compound are provided in therapeutically effective amounts.

15. A composition comprising Pro-2-PAM and the huperzine compound.

16. The composition of claim 15, wherein the composition comprises Pro-2-PAM and the huperzine compound in therapeutically effective amounts.

17. The composition of claim 16, wherein the therapeutically effective amounts are amounts which treat, prevent, inhibit, or reduce a seizure, an SE causing seizure, status epilepticus, neuropathogenesis, or a neuropathology caused by exposure to an organophosphate compound.

18. The method of claim 1, wherein the huperzine compound is a huperzine A compound, preferably +HupA.

19-22. (canceled)
